(19) United States
(12) Patent Application Publication
(10) Pub. No.: US 2026/0298615 A1
(43) Pub. Date: Oct. 1, 2026

(54) OPTICAL SENSOR MODULES WITH INTERFEROMETRIC SENSING

(71) Applicant: Apple Inc., Cupertino, CA (US)

(72) Inventors: Edward Vail, Menlo Park, CA (US); Thierry Oggier, Langnau am Albis (CH); Esther Chen, Santa Clara, CA (US); Fred Y. Chou, Cupertino, CA (US); Joseph R. Lee, Millbrae, CA (US)

(21) Appl. No.: 19/572,148

(22) Filed: Mar. 19, 2026

Related U.S. Application Data
(60) Provisional application No. 63/778,168, filed on Mar. 26, 2025.

(51) Int. Cl. G01B 9/02; G01B 9/02002; G02B 6/34; G06F 1/16; H05K 1/183

(52) U.S. Cl. CPC G01B 9/02045; G01B 9/02002; G02B 6/34; G06F 1/163; H05K 1/183; H05K 2201/10106; H05K 2201/10121; H05K 2201/10151

(57) ABSTRACT

Embodiments are directed to optical sensor modules having a sensor arrangement that includes at least one laser doppler interferometry (LDI) sensor. The LDI sensor(s) of an optical sensor module may be incorporated into a photonic printed circuit board, such that light may be routed within a plane of the photonic printed circuit board. In some instances, the sensor arrangement may include additional sensors, such as photoplethysmography (PPG) sensors, temperature sensors, combinations thereof, or the like.

### CROSS-REFERENCE TO RELATED APPLICATIONS

This application is a nonprovisional and claims the benefit under 35 U.S.C. 119(e) of U.S. Provisional Patent Application No. 63/778,168, filed Mar. 26, 2025, the contents of which are incorporated herein by reference as if fully disclosed herein.

### FIELD

The described embodiments relate generally to optical sensor modules, and electronic devices that incorporate these optical sensor modules, that incorporate one or more interferometric sensors. More specifically, the described embodiments relate to optical sensor modules that incorporate a photonic printed circuit board.

### BACKGROUND

Electronic devices, including wearable devices like smartwatches, head-mounted devices, and the like, increasingly include non-invasive sensing technologies that allow an electronic device to measure one or more physiological parameters of a user and/or information about the environment surrounding the electronic device. By measuring one or more physiological parameters, a user may gain insight into aspects into their health and wellness. Similarly, information about the environment surrounding an electronic device may provide information about how a device is being worn or held by a user. Electronic device may have limited available space, and thus it may be useful to provide additional sensing capabilities for an electronic device while maintaining a relatively small form factor.

### SUMMARY

Embodiments described herein are directed to optical sensor modules that include laser doppler interferometry sensors. Some embodiments are directed to an optical sensor module that includes a photonic printed circuit board having a first side and a second side. The optical sensor module may include a laser doppler interferometry sensor and a photoplethysmogram sensor. The laser doppler interferometry sensor includes a light source assembly configured to generate signal light and reference light, wherein the laser doppler interferometry is configured to emit the signal light from the first side of the photonic printed circuit board and collect return signal light through the first side of the photonic printed circuit board. The laser doppler interferometry sensor further includes an interferometer configured to receive the reference light and the return signal light and generate a pair of interference signals and a pair of photodetectors positioned to measure the pair of interference signals. The photoplethysmogram sensor includes a photodetector positioned on the first side of the first side of the photonic printed circuit board.

In some variations, the pair of photodetectors are positioned on the first side of the photonic printed circuit board. Alternatively, the pair of photodetectors are positioned on the second side of the photonic printed circuit board. In some variations, the photoplethysmogram sensor includes a light source positioned on the first side of the photonic printed circuit board. In other variations, the light source assembly comprises a coherent light source and the photoplethysmogram sensor includes the coherent light source. Additionally or alternatively, the photonic printed circuit board may include an optical routing layer, a first outcoupler positioned to couple the signal light from the optical routing layer toward the first side, and a second outcoupler positioned to couple the return signal light into the optical routing layer. In some of these variations, the first outcoupler is a prism outcoupler. Additionally or alternatively, the optical routing layer may include a prism interferometer that defines the second outcoupler and the interferometer.

Other embodiments are directed to an optical sensor module that includes a photonic printed circuit board defining a common waveguide, a common light source mounted to the photonic printed circuit board, and a plurality of laser doppler interferometry sensors. Each laser doppler interferometry sensor of the plurality of laser doppler interferometry sensors includes the common waveguide, the common light source, a corresponding set of waveguides optically connected to the common waveguide, a corresponding interferometer, and a corresponding pair of photodetectors.

In some of these variations, the corresponding interferometer for each laser doppler interferometry sensor of the plurality of laser doppler interferometry sensors is a prism interferometer. Additionally or alternatively, the common waveguide is a first common waveguide, the photonic printed circuit board defines a second common waveguide, and each laser doppler interferometry sensor of the plurality of laser doppler interferometry sensors includes a corresponding waveguide optically connected to the second common waveguide.

Still other embodiments are directed to an optical sensor module that includes a cover, an optical barrier defining an inner cavity and an outer cavity surrounding the inner cavity, and a photonic printed circuit board. The printed circuit board is configured to emit light from a first side of the photonic printed circuit board through a first set of optical apertures positioned in the inner cavity, and is configured to receive light received at the first side of the photonic printed circuit board through a second set of optical apertures positioned in the outer cavity. The optical sensor module further includes a set of light sources positioned on the first side of the photonic printed circuit board in the inner cavity and a first set of photodetectors positioned on the first side of the photonic printed circuit board in the outer cavity.

In some of these variations, the first set of optical apertures includes a plurality of optical apertures. Additionally or alternatively, the photonic printed circuit board is configured to emit a first wavelength of light through the first set of optical apertures, and the set of light sources is configured to emit a second wavelength of light. In some of these variations, the first set of photodetectors is configured measure the second wavelength of light. The optical sensor module may further include a second set of photodetectors positioned on the first side of the photonic printed circuit board in the outer cavity, wherein the second set of photodetectors is configured measure the first wavelength of light.

In some instances, the first set of photodetectors and the second set of optical apertures are radially arranged around the optical barrier. In some of these variations, photodetectors of the first set of photodetectors alternate with the optical apertures of the second set of optical apertures. The optical sensor module may include set of laser doppler interferometry sensors, wherein each laser doppler interferometry sensor of the set of laser doppler interferometry sensors includes: one or more corresponding optical apertures of the first set of optical apertures, a corresponding optical aperture of the second set of optical apertures, a corresponding interferometer, and a corresponding pair of photodetectors. In some of these variations, corresponding pair of photodetectors for each laser doppler interferometry sensor of the set of laser doppler interferometry sensors is positioned on a second side of the photonic printed circuit board.

In addition to the example aspects and embodiments described above, further aspects and embodiments will become apparent by reference to the drawings and by study of the following description.

### BRIEF DESCRIPTION OF THE DRAWINGS

The disclosure will be readily understood by the following detailed description in conjunction with the accompanying drawings, wherein like reference numerals designate like structural elements, and in which:

FIGS. 1A and 1B show a front and rear views, respectively, of an example electronic device that includes an optical sensor module such as described herein. FIG. 1C shows an exploded perspective view of the electronic device of FIGS. 1A and 1B.

FIG. 2 shows a schematic diagram of a variation of an optical sensor module, as described herein, that includes a laser doppler interferometry sensor.

FIG. 3A shows a top view of a variation of photonic printed circuit board, as described herein, that includes a sensor array. FIG. 3B shows a cross-sectional side view of the photonic printed circuit board of FIG. 3A, taken along line 3B-3B. FIG. 3C shows a partial bottom view of the photonic printed circuit board of FIG. 3A.

FIG. 4A shows a top view of a variation of a photonic printed circuit board, as described herein, that includes a sensor array. FIG. 4B shows a cross-sectional side view of the photonic printed circuit board of FIG. 4A, taken along line 4B-4B.

FIG. 5 shows a cross-sectional side view of a portion of a photonic printed circuit board, as described herein, that includes prism outcouplers.

FIG. 6A shows a partial top view of a variation of a photonic printed circuit board, as described herein, that includes a laser doppler interferometry sensor having a prism interferometer. FIG. 6B shows a cross-sectional side view of the photonic printed circuit board of FIG. 6A, taken along line 6B-6B.

FIGS. 7A and 7B shows top views of variations of sensor arrays, as described herein, that include a plurality of laser doppler interferometry sensors.

FIG. 8 shows a top view of a variations of a sensor array, as described herein, that include a plurality of laser doppler interferometry sensors.

FIGS. 9 and 10 show top views of variations of optical sensor modules, as described herein, that include a sensor array having both laser doppler interferometry and photoplethysmography sensors.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto. It should also be understood that in figures that show cross-sectional side views, certain components (e.g., waveguides, prisms) may be illustrated without hatching to aid in visualization of the overall optical sensor module (e.g., to illustrate the trajectory of light within the optical sensor module).

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The following disclosure relates optical sensor modules having a sensor arrangement that includes at least one laser doppler interferometry (LDI) sensor. The LDI sensor(s) of an optical sensor module may be incorporated into a photonic printed circuit board, such that light may be routed within a plane of the photonic printed circuit board. In some instances, the sensor arrangement may include additional sensors, such as photoplethysmography (PPG) sensors, temperature sensors, combinations thereof, or the like. Example optical sensor modules, as well as sensor arrangements that may be incorporated into an optical sensor module, are described in more detail herein.

Non-invasive cardiovascular monitoring may allow for the collection of real-time data associated with a user’s heart function and blood flow. Many optical sensor modules are configured to perform a PPG measurement, in which a measured signal varies as a function of change in blood volume. PPG measurements may be used to detect the timing of individual heartbeats, which may be used to calculate certain metrics associated with a users heartbeat (e.g., a user’s heart rate, heart rate variability, or the like) or identify certain patterns in a user’s heartbeat (e.g., sinus rhythm, an irregular rhythm, or the like). In some instances, PPG measurements may be used in measuring other physiological parameters of a user, such as respiration, blood pressure, or the like. PPG measurements may be susceptible to motion artifacts, ambient light, and low perfusion in tissue being measured as part of a PPG measurement. Accordingly, it may be preferably to perform a PPG measurement while an optical sensor module is in contact with a user’s skin, which may improve the accuracy of physiological parameters calculated using the PPG measurement.

In some instances, it may be desirable for an optical sensor module to be able to determine blood flow velocity. For example, an optical sensor module may be configured to perform a laser doppler flowmetry (LDF) measurement in which a measured signal varies as a function of change in blood flow velocity. An LDF measurement may provide additional information about a user’s cardiovascular state, and LDF measurements may also be used in determining physiological parameters such as respiration, blood pressure or the like. Because LDF measurements may be relatively insensitive to ambient light, LDF measurements may still be performed even when an electronic device is not in direct contact with a user’s skin. For example, an LDF measurement may be utilized as part of a bioauthentication process to help determine that live tissue is being measured (e.g., as part of a fingerprint recognition or facial recognition determination). To perform a LDF measurement, an optical sensor module may include one or more LDI sensors, such as described in more detail herein.

The electronic devices described herein include optical sensor modules that are configured to perform a LDF measurement. In some of these electronic devices, the optical sensor module is configured to perform both LDF and PPG measurements. In some instances, LDF and PPG measurements performed by an optical sensor module may both be used in determining a physiological parameter such as respiration rate, blood pressure, or the like. In these instances, using both LDF and PPG measurements may provide for more accurate determination of a physiological parameter as compared to using only LDF measurements or only PPG measurements.

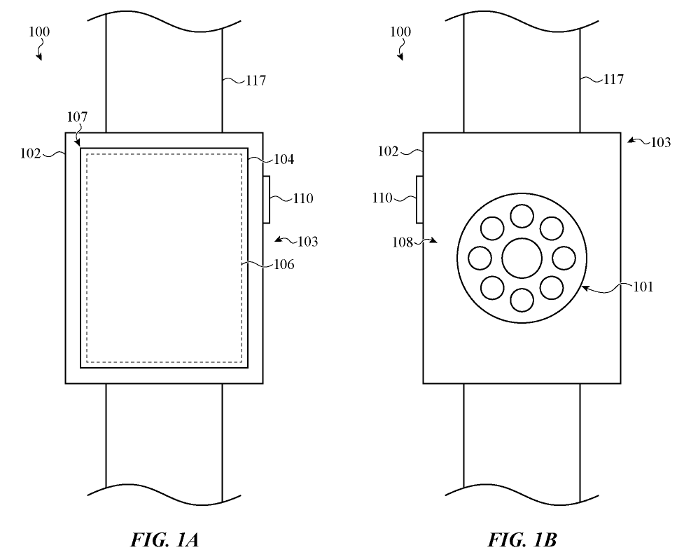

These and other embodiments are discussed below with reference to FIGS. 1A-10. However, those skilled in the art will readily appreciate that the detailed description given herein with respect to these figures is for explanatory purposes only and should not be construed as limiting.

FIGS. 1A and 1B show front and rear views, respectively, of an electronic device 100, as described herein. The electronic device 100 may be a portable electronic device such as, for example, a wearable electronic device (e.g., a watch, head-mounted device, or the like), a health monitoring device, a mobile phone, a portable or tablet computer, or the like. In the variation shown in FIGS. 1A and 1B, the electronic device 100 is configured as a watch, which is sometimes referred to as a smartwatch.

The electronic device 100 includes an optical sensor module 101, such as described in more detail herein, and a housing 102. FIG. 1C shows an exploded perspective view of the electronic device 100, which illustrates the components of the optical sensor module 101. The optical sensor module 101 and the housing 102 may collectively at least partially define an enclosure 103 of the electronic device 100. The enclosure 103 may house various components of the electronic device 100. The housing may be formed from a single piece or formed from multiple individual pieces as may be desired. In some embodiments, at least a portion of the housing 102 is electrically conductive. For example, one or more portions of the housing 102 may be formed of an electrically conductive material (e.g., a metal, a metal alloy, or the like).

In some variations, the electronic device 100 further includes a front cover 104 that may at least partially define the enclosure 103 (e.g., in combination with the optical sensor module 101 and the housing 102). The front cover 104 is positioned over a display 106 (the boundaries of which are indicated by dashed lines in FIG. 1A) that may be attached to (or may abut) the front cover 104. The display 106 may be configured to produce a graphical output that is visible to a user through a transparent portion of the front cover 104. For example, the display 106 may be configured as a liquid-crystal display (LCD), a light-emitting diode (LED) display, an LED-backlit LCD display, an organic light-emitting diode (OLED) display, an active layer organic light-emitting diode (AMOLED) display, or the like. In some cases, the display 106 may be configured as a touch-sensitive display that is configured to detect a touch input provided on the front cover 104. The display 106 may also include a display controller that is configured to control the operation of the display 106.

Overall, the electronic device 100 defines an exterior that includes a front surface 107 and a rear surface 108 that face in opposite directions. The exterior of the electronic device 100 may include at least one side surface 109 that connects the front surface 107 to the rear surface 108. The exterior may include a single side surface or multiple side surfaces connecting the front surface 107 to the rear surface 108, as may be desired. In the variation of the electronic device 100 shown in FIGS. 1A-1C, the front cover 104 (in combination with any exterior surface coatings) at least partially defines the front surface 107 of the electronic device 100 and the optical sensor module 101 at least partially define the rear surface 108 of the electronic device 100. In these variations, the optical sensor module 101 may perform optical measurements through the rear surface 108 of the electronic device 100 and the display 106 may produce a graphical output through the front surface 107 of the electronic device 100.

The electronic device 100 may include a strap 117 or other fastening mechanism that is configured to secure the electronic device 100 to a user. The strap 117 may be formed from a single piece or may include multiple pieces, and may be configured to at least partially wrap around a portion of a user’s body. For example, when the electronic device 100 is configured as a smartwatch, the electronic device 100 may be worn such that the strap 117 and housing 102 at least partially wrap around a user’s wrist, and the strap 117 may hold the housing 102 against the skin of the user’s wrist.

The electronic device 100 may further include one or more input mechanisms, such as an input mechanism 110 shown in FIGS. 1A and 1B, that are configured to receive a physical input from a user. Each input mechanism may be configured, for example, as a push button, a touch-activated button (e.g., a capacitive touch button), a crown, a dial, or the like. Each input mechanism may provide one or more functions, such that a user may manipulate the input mechanism to initiate or otherwise control a corresponding function of the electronic device 100. For example, a user may initiate a measurement performed by the optical sensor module 101 by manipulating the input mechanism 110. Additionally or alternatively, the electronic device 100 may be configured to operate the display 106 to present a graphical output, in response to receiving an input at the input mechanism 110, that may include information about (e.g., the results of) of a measurement performed by the optical sensor module 101.

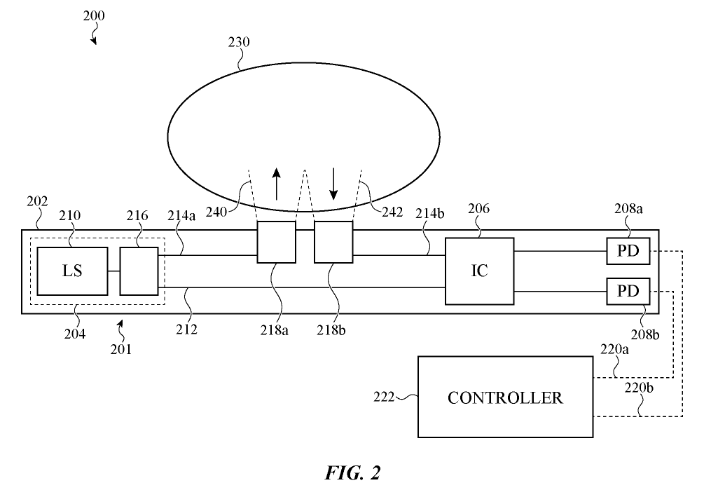

FIG. 1C illustrates the components of the optical sensor module 101. Specifically, the optical sensor module 101 may include a cover 112 and a photonic printed circuit board (PCB) 114. The photonic PCB 114 includes sensor array 116 that includes at least one LDI sensor, such as described in more detail herein. The sensor array 116 may include a single LDI sensor or a plurality of LDI sensors as may be desired. In some variations, the sensor array 116 also includes at least one PPG sensor. The sensor array 116 may include a single PPG sensor or a plurality of PPG sensors as may be desired. Examples of photonic PCBs and associated sensor arrays are described in more detail herein, such as in relation to FIGS. 3A-10.

The cover 112 may be attached to the housing 102 and may thus define a corresponding portion of the enclosure 103 of the electronic device 100. For example, the housing 102 may define a rear opening 118 that extends through the housing 102, and the cover 112 may be positioned to cover the rear opening 118. Accordingly, the cover 112 may enclose the photonic PCB 114 and other components of the optical sensor module 101 within the enclosure 103 of the electronic device. The cover 112 is shown in FIG. 1C as having a circular perimeter, though it should be appreciated that the perimeter of the cover 112 may have any shape (e.g., square, oval, or the like) as may be desired.

The cover 112 may define one or more windows, each of provides an on optical path through which light may pass through the cover 112. For example, the cover 112 may define one or more windows (also referred herein to as “collection windows”) through which light may enter the optical sensor module 101 and be measured by the sensor array 116. Similarly, the cover 112 may define one or more windows (also referred to herein as “launch windows”) through which generated by the sensor array 116 may exit the optical sensor module 101. In this way, the sensor array 116 may emit light from the electronic device 100 through the cover 112 (e.g., via one or more launch windows) and may collect that is returned the sensor array 116 through the cover 112 (e.g., via one or more collection windows). For example, when the electronic device 100 is positioned such that cover 112 faces a user’s skin (e.g., is positioned such that the cover contacts the user’s skin), the sensor array 116 may, as part of an optical measurement (e.g., a PPG measurement or a LDF measurement as described herein), emit light toward the user’s skin and may collect and measure light that returns to the electronic device 100 after interacting with the user’s skin.

In the variation shown in FIG. 1C, the cover 112 defines a plurality of collection windows 120a-120h that are radially positioned around a launch window 122. This is just one example, and it should be appreciated that the photonic PCBs and associated sensor arrays may be used covers having different configurations of windows. For example, the cover 112 may include a single collection window or a plurality of collection windows and/or a single launch window or a plurality of launch windows. It should also be appreciated that a window of the cover 112 may act as both a collection window and a launch window, such that the sensor array 116 may emit and collect light through the same window of the cover 112.

The photonic PCB 114 may be positioned within the electronic device 100 behind the cover 112. For examples, the photonic PCB 114 may be attached the cover 112, either directly or via one or more intervening structures. In some variations, the optical sensor module 101 includes a set of optical barriers 124a-124b that connects the photonic PCB 114 to the cover 112. The set of optical barriers 124a-124b may limit the pathways along which light may travel within the optical sensor module 101, and may be formed form a material that is opaque to the wavelength(s) of light that are emitted by the sensor array 116. Accordingly, the set of optical barriers 124a-124b may reduce the measurement of stray light by the sensor array 116.

For example, in the variation shown in FIG. 1C, the set of optical barriers 124a-124b includes a first optical barrier 124a and a second optical barrier 124b that surrounds the first optical barrier 124a. The set of optical barriers 124a-124b may define a first cavity 126a (e.g., surrounded by the first optical barrier 124a) and a second cavity 126b (e.g., between the first optical barrier 124a and the second optical barrier 124b) within the optical sensor module 101, such that the first cavity 126a is optically isolated from the second cavity 126b. In these instances, the sensor array 116 may emit light that passes through the first cavity 126a before exiting the optical sensor module 101 via the launch window 122, and may similarly collect light that passes through second cavity 126b when it enters the optical sensor module 101 via one or more of the collection windows 120a-120h. The first optical barrier 124a may limit the ability of light emitted by the sensor array 116 to be measured by the sensor array 116 without first exiting the optical sensor module 101 via the cover 112. While the first optical barrier 124a and the second optical barrier 124b are shown in FIG. 1C as having concentric annular shapes, it should be appreciated that the optical sensor modules described herein may include any number of arrangements of optical barriers as may be desired.

In some examples, the optical sensor module 101 may further include a controller (not shown). The controller may include any suitable combination of hardware, software, and/or firmware as may be necessary to control the various operations of the optical sensor module 101. For example, the controller may operate one or more light sources of the sensor array 116 to generate light as part of a given optical measurement, and may operate one or more photodetectors of the sensor array 116 to measure light as part of the optical measurement.

In some examples, the optical sensor controller may include memory and one or more processors. Memory can include one or more non-transitory computer-readable storage mediums for storing computer-executable instructions, which, when executed by one or more computer processors can cause the computer processors to control the sensor array 116 to perform one or more optical measurements as described herein. A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. A non-transitory computer-readable storage medium may include, but is not limited to, magnetic, optical, and/or semiconductor storages. Examples of such storage include magnetic disks, optical discs, as well as persistent solid-state memory such as flash, solid-state drives, and the like. The one or more processors may be any component capable of processing, receiving, or transmitting data or instructions, such as a microprocessor, central processing unit (CPU), an application-specific integrated circuit (ASIC), a digital signal processor (DSP), a controller, or a combination of such components.

It should be appreciated that different components of the controller may be distributed throughout the electronic device 100. It should also be appreciated that while the controller may control and operate the optical sensor module 101 to perform one or more optical measurements, the resulting measurement signals generated by the optical sensor module 101 may undergo one or more signal processing operations or analysis by other processing circuitry. For example, the controller may transmit measurement signals generated by the optical sensor module 101 to another processor of the electronic device 100 for further processing or analysis. Additionally or alternatively, the electronic device 100 may transmit (e.g., using one or more wireless communication techniques) the measurement signals generated by the optical sensor module 101 to another electronic device for further processing or analysis.

As described herein, an LDI sensor includes a light source assembly, an interferometer, and a pair of photodetectors. The light source assembly includes a coherent light source that is configured to generate light along two optical paths. A first optical path includes a first portion of the light generated by the coherent light source (also referred to herein as “signal light”) and a second optical path includes a second portion of the light generated by the coherent light source (also referred to herein as “reference light”). The LDI sensor is configured to emit the signal light and collect light that is returned to the LDI sensor. For the purpose of discussion, the signal light that is emitted from an LDI sensor is referred to herein as “emitted signal light” and the light is collected by the LDI sensor is referred to herein as “return signal light.” It should be appreciated that, in practice, the return signal light may include both i) a portion of the emitted signal light that is returned to the LDI sensor after interacting with the external environment (e.g., a user’s skin) and ii) ambient light that reaches the LDI sensor. The return signal light and the reference light may be received by the interferometer, which interferes the return signal light and the reference light to generate a pair of interference signals. Each interference signal includes light with an intensity that depends on the interference between the return signal light and the reference light. The pair of photodetectors may receive and measure the pair of interference signals to generate a set of electrical LDF measurement signals. The LDF measurement signals may be analyzed to determine information about a user’s blood flow velocity.

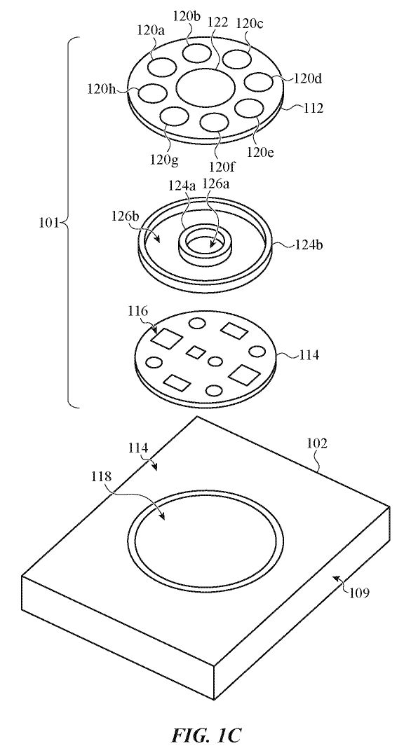

FIG. 2 shows a schematic view of an optical sensor module 200 that includes a photonic PCB 202 and an LDI sensor 201 incorporated into the photonic PCB 202. Specifically, the LDI sensor 201 includes a light source assembly 204, an interferometer 206, and a pair of photodetectors 208a-208b. The light source assembly 204 includes a coherent light source 210, such as a laser, that is configured to generate coherent light. The coherent light source 210 may be single-frequency (fixed wavelength) or may be tunable to selectively generate one of multiple wavelengths (e.g., the light source may be controlled to output different wavelengths at different times). Examples of coherent light sources 210 include, but are not limited to, distributed feedback lasers (DFBs), vertical cavity surface-emitting lasers (VCSELs), vertical-external-cavity surface-emitting lasers (VECSELs), edge-emitting lasers (EELs), horizontal cavity surface-emitting lasers (HCSELs), quantum dot lasers (QDLs), quantum cascade laser (QCLs), or the like.

The light source assembly 204 is configured to generate light that is carried along two optical paths within the photonic PCB 202. Specifically, the photonic PCB 202 includes a reference path 212 and a set of signal paths 214a-214b that optically connect the light source assembly 204 to the interferometer 206. Accordingly, the light source assembly 204 may be configured to generate signal light and reference light. In some variations, the light source assembly 204 includes a splitter 216 that is configured to split light generated by the coherent light source 210 into a first portion that forms the signal light and a second portion that forms the reference light. The splitter 216 may be any suitable component configured to split light received an input thereof, such as a y-branch splitter, a directional coupler, a multimode interference splitter, or the like.

In some variations, the splitter 216 may be asymmetric, such that the splitter 216 divides light with an unequal power distribution. For example, the light source assembly 204 may be configured such that the signal light has a higher intensity than the reference light. Accordingly, the splitter 216 may split light according to an uneven splitting ratio to provide a relatively higher intensity for the signal light than the reference light. In one non-limiting example, the splitter 216 may have a 95/5 splitting ratio, such that the 95% of the light generated by the coherent light source 210 is routed to the set of signal paths 214a-214b as signal light and 5% of the light generated by the coherent light source 210 is routed to the reference path 212 as reference light.

In other variations, the coherent light source 210 may be configured to generate multiple light outputs. Specifically, some coherent light sources may be configured to simultaneously generate two different beams of light. For example, a DFB laser may be configured to simultaneously emit light from two different facets. In these variations, a first light output generated by a coherent light source may be received by a set of signal paths as signal light, and a second light output generated by the coherent light source may be received by a reference path as reference path. Accordingly, the light source assembly may not include the splitter 216 in these instances, as the signal light and reference light are generated as different outputs of the coherent light source 210.

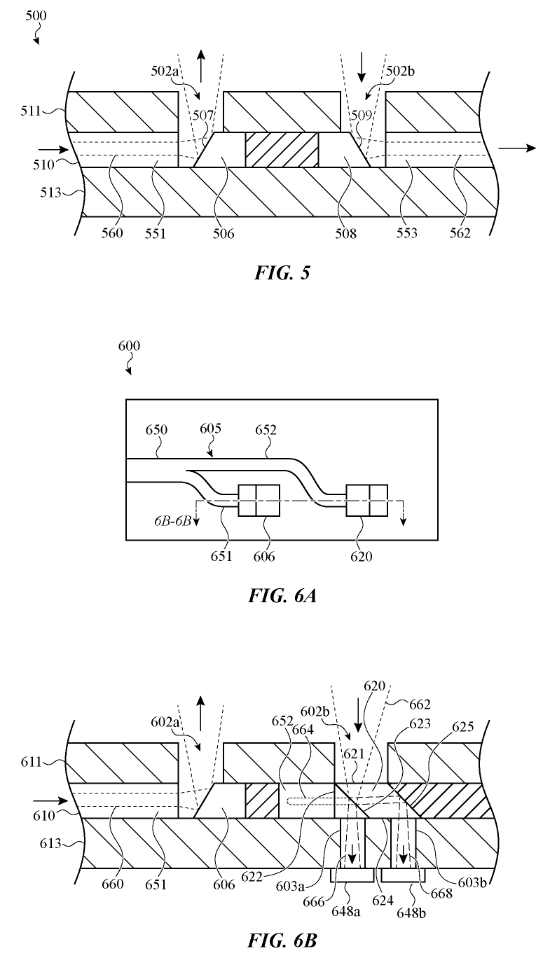

The LDI sensor 201 further includes a set of outcouplers 218a-218b that couple light into and out of the photonic PCB 202. Specifically, the set of signal paths 214a-214b includes a first signal path 214a that optically connects the light source assembly 204 to a first outcoupler 218a of the set of outcouplers 218a-218b. Accordingly, signal light generated by the light source assembly 204 may be routed to the first outcoupler 218a, and the first outcoupler 218a may emit this signal light from the photonic PCB 202 as emitted signal light 240. Similarly, a second signal path 214b optically connects a second outcoupler 218b of the set of outcouplers 218a-218b to a first input of the interferometer 206. The second outcoupler 218b collects return signal light 242 and couples the return signal light 242 into the second signal path 214b, such that the interferometer 206 may receive the return signal light 242 at its first input. It should be appreciated that in some instances, a single component (e.g., a prism interferometer such as described herein with respect to FIGS. 6A and 6B) may act as both the second outcoupler 218b and the interferometer 206.

The reference path 212 optically connects the light source assembly 204 to a second input of the interferometer 206. As such, the interferometer 206 receives return signal light 242 at its first input along the second signal path 214b and receives reference light at its second input along the reference path 212. The interferometer 206 may interfere the reference light with the return signal light and may output a pair of interference signals that includes a first interference signal and a second interference signal. Specifically, the interferometer 206 may include a first output that outputs the first interference signal and a second output that outputs the second interference signal. In some instances, the interferometer 206 may include or otherwise be optically connected to a set of one or more outcouplers that redirect the first and second interference signals toward respective photodetectors of the pair of photodetectors 208a-208b.

The pair of photodetectors 208a-208b are positioned to measure the pair of interference signals. Specifically, the pair of photodetectors 208a-208b includes a first photodetector 208a that is optically connected to the first output of the interferometer 206, such that the first photodetector 208a receives and measures the first interference signal. The pair of photodetectors 208a-208b further includes a second photodetector 208b that is optically connected to the second output of the interferometer 206, such that the second photodetector 208b receives and measures the second interference signal. The pair of photodetectors 208a-208b may output a set of LDF measurement signals 220a-220b, which may be received by a controller 222 associated with the optical sensor module 200.

The controller 222 may analyze the set of LDF measurement signals 220a-220b to determine velocity information measured by the LDI sensor 201. Specifically, the controller 222 may be configured to analyze a signal that represents the difference between light measured by the first photodetector 208a and light measured by the second photodetector 208b (also referred to herein as a “differential LDF measurement signal”). The differential LDF measurement signal may be generated digitally or electrically. In some variations, the controller 222 receives two electrical measurement signals from the pair of photodetectors 208a-208b, including a first LDF measurement signal 220a received from the first photodetector 208a and a second LDF measurement signal 220b received from the second photodetector 208b, and may convert these signals to digital signals (e.g. using an analog-to-digital converter). In these variations, the controller 222 may digitally generate a differential LDF measurement signal by taking a difference between the digitized first LDF measurement signal 220a and the digitized second LDF measurement signal 220b. In other variations, the LDI sensor 201 may include a differential amplifier or other circuitry that receives the first LDF measurement signal 220a and the second LDF measurement signal 220b and generates the differential LDF measurement signal electrically. In these variations, the controller 222 may receive and digitize the electronic differential LDF measurement signal.

The optical sensor module 200 may be positioned such that light emitted by the LDI sensor 201 (e.g., the emitted signal light 240) enters the skin of a user (e.g., the wrist 230 of a user as depicted in FIG. 2). In these instances, the return signal light 242 may include photons from the emitted signal light 240 that have interacted with the user’s skin and returned to the LDI sensor 201. When a photon interacts with a moving target, such as blood flowing in the user’s skin, the frequency of the photon may be changed due to the Doppler effect. A given change in frequency in the return signal light 242 may, when interfered with the reference light, may generate an optical signal having a corresponding frequency. Accordingly, if a single frequency of light were returned to the LDI sensor 201 as return signal light 242 (and assuming the frequency of the emitted signal light and the reference light remain constant), the differential LDI measurement signal would vary with a corresponding frequency that depends on the frequency of the returned signal light.

In practice, however, the return signal light 242 includes multiple photons that are Doppler shifted by different amounts. For example, photons that are returned after relatively shallow penetration into a user’s skin may interact with shallower blood vessels (e.g., capillaries) in which blood flow is relatively slower. These photons may experience a relatively small frequency shift. Conversely, photons that are returned after relatively deeper penetration into a user’s skin may interact with deeper blood vessels (e.g., larger arteries or veins) in which blood flows relatively faster. These photons may experience a relatively large frequency shift. Accordingly, by looking at the frequency content of a differential LDF measurement signal (e.g., using a Fourier transform to convert the LDF measurement signal to the frequency domain), the controller 222 may estimate the relative amount of light received as a function of frequency shift. This may, in turn, indicate the distribution of blood flow velocities that are measured by the LDI sensor 201. The distribution of blood flow velocities, when monitored over time, may provide information about changes in a user’s blood flow. Additionally, because the differential LDF measurement signal is based on the difference between the outputs of the pair of photodetectors 208a-208b, this signal may be relatively insensitive to changes in ambient light. Accordingly, the controller 222 may determine blood flow information about a user in a wide range of potential ambient lighting conditions.

When a sensor, such as the LDI sensor 201 of FIG. 2, is incorporated in a photonic PCB, the photonic PCB may be configured to both provide optical routing of light within the sensor as well as providing electrical connections to components of the sensors. Specifically, a photonic PCB may include a layer stack that includes a set of optical routing layers, a set of electrical routing layers, and a set of electrically insulating layers.

Each optical routing layer of the set of optical routing layers may define one or more optical paths that allow for light to be routed laterally within the photonic PCB. Specifically, the optical routing layer defines a set of waveguides for routing light within the optical routing layer. Each waveguide may include a waveguide core that is formed from an optically transparent material (e.g., transparent to at least the wavelengths of light that are generated and/or measured by the LDI sensor(s) of the photonic PCB) and may optically confine light within the plane of the optical routing layer. The waveguide core may be at least partially surrounded by a cladding material (e.g., a material having a lower refractive index than the waveguide core) that helps to optically confine light within the waveguide core. The set of waveguides may route light between different components of an LDI sensor (a light source of the LDI sensor and an interferometer of the LDI sensor). In some instances, such as described in more detail herein, one or more waveguides of the set of waveguides may be configured to operate as a splitter, an interferometer, or the like.

It should also be appreciated that, in some instances, an optical routing layer of the set of optical routing layers may also include additional materials that are coplanar with the set of waveguides. For examples, the optical routing layer may include a substrate material such as a composite material (e.g., a prepreg material such as FR-4), a ceramic material, polyimide or the like. The substrate material may at least partially surround the set of waveguides, and may provide mechanical support (and in some instances, rigidity) to the optical routing layer. In some of these instances, the optical routing layer may also act as an electrically insulating layer to provide insulation between different electrical routing layers of the set of electrical routing layers. Additionally or alternatively, the optical routing layer may include one or more conductive vias (e.g., through-hole vias, blind vias, and/or buried vias) that provide one or more corresponding electrical connections through the optical routing layer.

The set of electrical routing layers may be configured to route electrical signals within the photonic PCB. Specifically, each electrical routing layer may be formed from an electrically conductive material (e.g., copper, or the like) that is patterned or otherwise formed to define a set of electrical traces, as will readily be understood by someone of ordinary skill in the art. The set of electrical routing layers may facilitate the control and operation of components of a sensor array carried by the photonic PCB. For example, the set of electrical routing layers may electrically connect a light source to a controller, such that the controller may operate the light source to generate light. Similarly, the set of electrical routing layers may electrically connect a photodetector to the controller, such that controller may operate the photodetector to measure light received by the photodetector.

The set of insulating layers may provide electrical insulation and/or mechanical support to the photonic PCB. For example, in variations in which a photonic PCB includes multiple electrical routing layers, an insulating layer may be positioned between two electrical routing layers to electrically insulate the electrical routing layers from each other. Some or all of the set of insulating layers may include conductive vias that provide one or more corresponding electrical connections through the insulating layer. Each insulating layer may be formed from one or more dielectric materials, such as the substrate materials described herein with respect to the set of optical routing layers.

FIGS. 3A-3C depict a variation of a photonic PCB 300 that may be used with the optical sensor modules described herein. The photonic PCB 300 may include a planar layer stack that defines a first side 301 and a second side 303 opposite the first side 301 (e.g., the first side 301 and the second side 303 face opposite directions). Specifically, the layer stack may include a set of (e.g., one or more) optical routing layers, a set of (e.g., one or more) electrical routing layers, and a set of (e.g., one or more) insulating layers. For example, the photonic PCB 300 is depicted in FIG. 3B as having a layer stack that includes an optical routing layer 310, an electrical routing layer 312, and a plurality of insulating layers 314a-314c.

While the photonic PCB 300 is shown in FIG. 3B as including a single optical routing layer 310, it should be appreciated that in other variations the photonic PCB 300 may include a plurality of optical routing layers as may be desired. In the variation shown in FIG. 3B, the optical routing layer 310 is configured as an intermediate layer within the layer stack, such that the photonic PCB 300 includes a first set of layers that is positioned above the optical routing layer 310 (e.g., between the optical routing layer 310 and the first side 301 of the photonic PCB 300) and a second set of layers positioned below the optical routing layer 310 (e.g., between the optical routing layer 310 and the second side 303 of the photonic PCB 300). For example, in the variation shown FIG. 3B, the first set of layers includes a first insulating layer 314a of the plurality of insulating layers 314a-314c, a second insulating layer 314b of the plurality of insulating layers 314a-314c, and the electrical routing layer 312 positioned between the first and second insulating layers 314a, 314b, and the second set of layers includes a third insulating layer 314c of the plurality of insulating layers 314a-314c. In these variations, the first insulating layer 314a at least partially defines the first side 301 of the photonic PCB 300 and the third insulating layer 314c at least partially defines the second side 303 of the photonic PCB 300. It should be appreciated, however, that the optical routing layer 310 may be positioned at an end (e.g., at the bottom) of the layer stack of the photonic PCB 300. In these instances, the optical routing layer 310 may at least partially define the second side 303 of the photonic PCB 300.

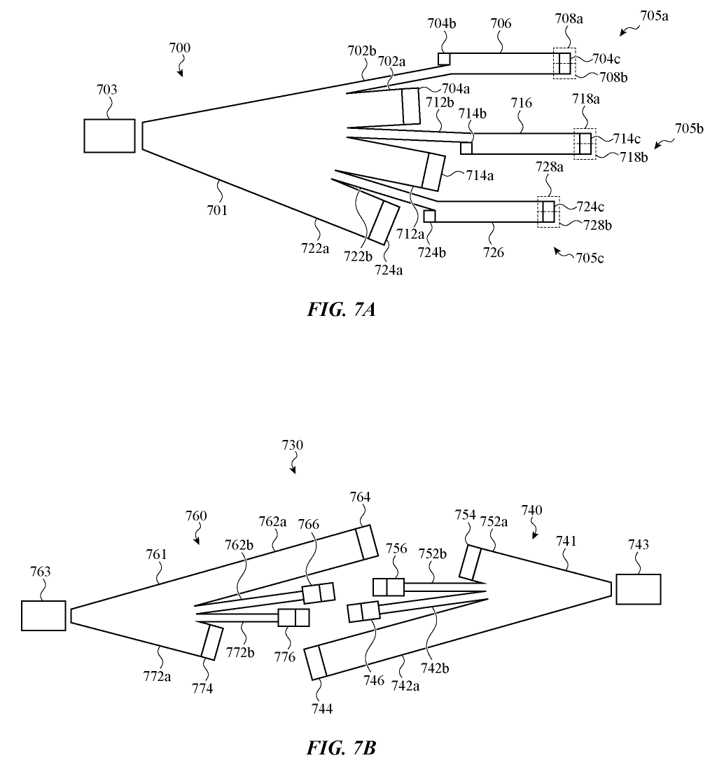

The optical routing layer 310 may route light that is generated and/or measured by a sensor of optical sensor module that incorporates the photonic PCB 300. For example, the photonic PCB 300 includes a sensor array that includes at least one LDI sensor 340 configured to perform LDF measurements. Although a single LDI sensor 340 is shown in the photonic PCB 300 of FIGS. 3A-3C, it should be appreciated that the sensor array may include a plurality of LDI sensors (such as described herein with respect to FIGS. 7A-10). Each LDI sensor 340 may include a corresponding light source assembly that includes a coherent light source 342. In some variations, the light source assembly may further include a splitter 344, such as shown in FIG. 3C. Additionally, each LDI sensor 340 may include a corresponding interferometer 346 and a corresponding pair of photodetectors 348a-348b.

The optical routing layer 310 may define a set of waveguides that may route light generated by the light source assembly of the LDI sensor 340, and may further include a plurality of outcouplers 360a-360c that are configured to couple light into or out of the optical routing layer 310. The set of waveguides may be configured to define one or more optical paths and/or photonic components (e.g., a splitter and/or interferometer) within the optical routing layer 310. In some instances where the LDI sensor 340 includes a splitter 344, the set of waveguides may be configured to define the splitter 344.

For example, FIG. 3C shows a bottom view of the photonic PCB 300 with the second set of layers (e.g., the third insulating layer 314c) removed to illustrate the set of waveguides defined in the optical routing layer 310. As shown in FIG. 3C, the set of waveguides includes a first waveguide 350 that is positioned to receive light that is generated by the coherent light source 342. Specifically, the first waveguide 350 may have a first end that is positioned adjacent to the coherent light source 342, such that light generated by the coherent light source 342 couples into the first end of the first waveguide 350. A second end of the first waveguide 350 may be connected each of a second waveguide 351 and a third waveguide 352, such that light traveling through the first waveguide 350 is split between the second waveguide 351 and the third waveguide 352. Accordingly, the first waveguide 350, the second waveguide 351, and the third waveguide 352 may collectively define the splitter 344.

The first waveguide 350 may accordingly receive light generated by the coherent light source 342 and may split that light into a first portion (e.g., signal light) that is received by the second waveguide 351 and a second portion (e.g., reference light) that is received by the third waveguide 352. The second waveguide 351 may be optically coupled to a first outcoupler 360a of the first plurality of outcouplers 360a, which may couple the light the signal light out of the optical routing layer 310 and out of the photonic PCB 300. Accordingly, the second waveguide 351 may act as a first signal path for the LDI sensor 340 to receive signal light, and the first outcoupler 360a may emit signal light from the photonic PCB 300.

To facilitate light exiting and entering the optical routing layer 310, the photonic PCB 300 may define one or more optical apertures that each extend through one or more layers of the layer stack of the photonic PCB 300. For example, in the variation of the photonic PCB 300 shown in FIGS. 3A-3C, the photonic PCB 300 includes a first set of optical apertures 302a-302b that extend through the first set of layers between the optical routing layer 310 and the first side 301 of the photonic PCB 300. The first set of optical apertures 302a-302b may include a plurality of optical apertures including a first optical aperture 302a and a second optical aperture 302b. Each of the first set of optical apertures 302a-302b represents a corresponding transparent region that extends between the optical routing layer 310 and the first side 301 of the photonic PCB 300. In this way, each of the first set of optical apertures 302a-302b acts as an optical via that allows light to travel between the optical routing layer 310 and the first side 301 of the photonic PCB 300. Each of the first set of optical apertures 302a-302b may be defined by a corresponding cavity that extends through the first set of layers (e.g., through each of the first and second insulating layers 314a, 314bof the plurality ofinsulatinglayers 314a-314c, and the electrical routing layer 312). The corresponding cavity may be at least partially filled with an optically transparent material or may be unfilled (e.g., such that air may be positioned within the cavity).

The first optical aperture 302a may be positioned over (e.g., above) the first outcoupler 306a, such that the first outcoupler 306a directs light out of the plane of the optical routing layer 310 and through the first optical aperture 302a. Accordingly, the LDI sensor 340 may emit the emitted signal light from the first side 301 of the photonic PCB 300 through the first optical aperture 302a. Similarly, the second optical aperture 302b may be positioned over (e.g., above) a second outcoupler 360b of the plurality of outcouplers 360a-360c. The LDI sensor 340 may, during an LDF measurement, collect the return signal light from the first side 301 of the photonic PCB 300 through the second optical aperture 302b, and the second outcoupler 360b may couple the return signal light into the optical routing layer 310.

In some variations, the second outcoupler 360b may be optically coupled to a fourth waveguide 353, such that light entering the photonic PCB 300 through the second optical aperture 302b is coupled into the fourth waveguide 353. In this way, the fourth waveguide 353 acts as a second signal path for the LDI sensor 340. When the coherent light source 342 is operated to generate light, the third waveguide 352 will receive reference light (and thereby act as a reference path for the LDI sensor 340) and the fourth waveguide 353 will receive return signal light. Each of the third waveguide 352 and the fourth waveguide 353 may be optically coupled to an interferometer.

For example, in the variation of the photonic PCB 300 shown in FIGS. 3A-3C, the set of waveguides includes a fifth waveguide 354 that operates as a waveguide interferometer and forms the interferometer for the LDI sensor 340. The fifth waveguide 354 has a first end optically coupled to each of the third waveguide 352 and the fourth waveguide 353. The fifth waveguide 354 may simultaneously receive the reference light and the return signal light from the third waveguide 352 and the fourth waveguide 353, respectively, and the reference light and return signal light may interfere while traveling through the fifth waveguide 354. The fifth waveguide 354 may be sized to support multiple modes of light, such that the fifth waveguide 354 acts as a multimode waveguide interferometer.

A second end of the fifth waveguide 354 may be optically coupled to a third outcoupler 360c of the plurality of outcouplers 360a-360c. In the variation of the LDI sensor 340 shown in FIGS. 3A-3C, the third outcoupler 360c is configured to direct light received from the fifth waveguide 354 toward the second side 303 of the photonic PCB 300. For example, the photonic PCB 300 may include a second set of optical apertures (shown in FIG. 3B as including a single optical aperture 304, also referred to herein as “third optical aperture 304”). The second set of optical apertures may be configured in any manner as described herein with respect to the first set of optical apertures 302a-302b, except that each of the second set of optical apertures extends through the second set of layers between the optical routing layer 310 and the second side 303 of the photonic PCB 300. The third optical aperture 304 may be positioned over (e.g., below) the third outcoupler 360c, such that the third outcoupler 360c couples light out of the optical routing layer 310 and through the third optical aperture 304. The pair of photodetectors 348a-348b may be positioned on the second side 303 of the photonic PCB 300, and may be positioned at least partially over the third optical aperture 304. Specifically, a first photodetector 348a of the pair of photodetectors 348a-348b is positioned at least partially over the third optical aperture 304, and will receive a first portion of the light redirected by the third outcoupler 360c. The first portion of the light redirected by the third outcoupler 360c may be a first interference signal with a first intensity that depends on the interference between the reference light and the return signal light. Similarly, a second photodetector 348b of the pair of photodetectors 348a-348b is positioned at least partially over the third optical aperture 304, and will receive a second portion of the light redirected by the third outcoupler 360c. The second portion of the light redirected by the third outcoupler 360c may be a second interference signal with a second intensity that depends on the interference between the reference light and the return signal light. Accordingly, the pair of photodetectors 348a-348b may measure a pair of interference signals as described in more detail herein.

The LDI sensor 340 of FIGS. 3A-3C is configured to use a single outcoupler (e.g., third outcoupler 360c) to redirect both the first and second interference signal from the optical routing layer 310 to the pair of photodetectors 348a-348b, but it should be appreciated that an LDI sensor 340 may be configured such that different outcouplers are used to output the first and second interference signals from the optical routing layer 310. For example, the output of the fifth waveguide 354 may be split along two different output waveguides. A first of these output waveguides may be optically connected to the third outcoupler 360c, and may route a first interference signal to the third outcoupler 360c. The third outcoupler 360c may direct the first interference signal to the first photodetector 348a through the third optical aperture 304. A second of these output waveguides may be optically connected to a fourth outcoupler (not shown), and may route a second interference signal to the fourth outcoupler. A fourth optical aperture (not shown) may be positioned over the fourth outcoupler, and the fourth outcoupler may direct the second interference signal to the second photodetector 348b through the fourth optical aperture.

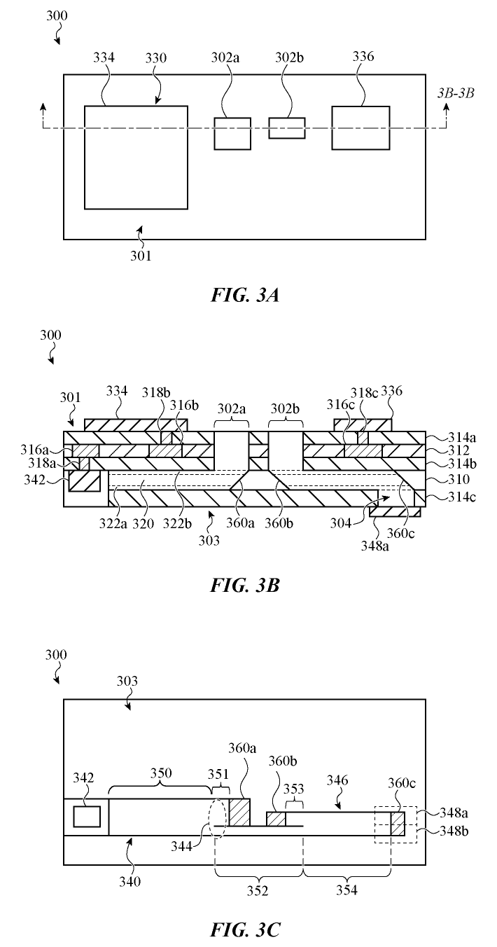

In the variation of the LDI sensor 340 shown in FIGS. 3A-3C, each photodetector of the pair of photodetectors 348a-348b is positioned on the second side 303 of the photonic PCB 300, which may allow for additional flexibility in positioning other components (e.g., additional sensors) on the first side 301 of the photonic PCB 300 and/or may allow for a smaller overall footprint of the photonic PCB 300. In other variations, one or both of the pair of photodetectors 348a-348b may be positioned on the first side 301 of the photonic PCB 300. For example, FIGS. 4A and 4B show a variation of a photonic PCB 400 that is configured and labeled the same except that the pair of photodetectors 348a-348b are positioned on the first side 301 of the photonic PCB 400 and the third optical aperture 304 has been replaced with a third optical aperture 404 that extends through the first set of layers. In these variations, the third outcoupler 360c is configured to redirect light toward the first side 301. Accordingly, the third outcoupler 360c couples light (e.g., the first and second interference signals) out of the optical routing layer 310 and through the first set of layers via the third optical aperture 404, such that the interference signals may be measured by the pair of photodetectors 348a-348b.

In some variations, the waveguide defined by in the optical routing layer may a waveguide core that is at least partially surrounding by a cladding material. The cladding material may have a lower refractive index than the waveguide core, which may help to confine light within the waveguide core. For example, in the variation of the photonic PCB 300 shown in FIGS. 3A-3C, the optical routing layer 310 may include a waveguide core 320 that is positioned between a lower cladding layer 322a and an upper cladding layer 322b. Accordingly, the lower cladding layer 322a may be positioned between the waveguide core 320 and the second side 303 of the photonic PCB 300, and the upper cladding layer 322b may be positioned between the waveguide core 320 and the first side 301 of the photonic PCB 300. The waveguide core 320 may be formed form a plastic material, such as polycarbonate, polymethyl methacrylate, or the like. The cladding material may also be formed from a plastic material, such as polyacrylate or the like. The waveguide core 320 and the upper and lower cladding layers 322a, 322b may have any suitable relative thicknesses along a vertical direction of the photonic PCB 300. For example, in some variations, the waveguide core 320 may have a thickness that is at least twice the corresponding thickness of each of the upper and lower cladding layers 322a, 322b. In one non-limiting example, the waveguide core 320 may have a thickness of 50 µm, and each of the upper and lower cladding layers 322a, 322b may have a thickness of 25 µm.

In the variation of the photonic PCB 300 shown in FIGS. 3A-3C, the sensor array includes at least one PPG sensor 330 configured to perform PPG measurements. Specifically, each PPG sensor 330 includes a corresponding photodetector 334 mounted to the first side 301 of the photonic PCB 300 and a light source 336. During a PPG measurement, the light source 336 is configured to emit light from the first side 301 of the photonic PCB 300 and the photodetector 334 is configured to measure light that is returned to the photonic PCB 300 (e.g., after interacting with the external environment, such as a user’s skin). The amount of light measured by the photodetector 334 during a PPG measurement may depend, at least in part, on the distance between the photonic PCB 300 and a user’s skin, the amount of ambient light present, as well as the relative amount of blood in the skin that is measured by the PPG sensor 330. Accordingly, a PPG measurement performed by the PPG sensor 330 may be used to measure changes in blood volume of a user’s skin (which may be used to measure heart rate, such as described in more detail herein) and/or a distance between the PPG sensor 330 and the user’s skin.

In some instances, the PPG sensor 330 may include a light source (e.g., light source 336) that is different from the coherent light source 342 of the LDI sensor 340. For example, in some variations the PPG sensor 330 may include a light source 336 that is mounted to the first side 301 of the photonic PCB 300 and that is configured to emit light away from the first side 301 of the photonic PCB 300. In some of these variations, the light source 336 may include a light emitting diode. In some variations in which the LDI sensor 340 and the PPG sensor 330 include different light sources, these light sources may be configured to emit different wavelengths of light. For example, the coherent light source 342 of the LDI sensor 340 may be configured to emit a first wavelength of light, and the light source 336 of the PPG sensor 330 may be configured to emit a second wavelength of light that is different than the first wavelength of light.

In some of these variations, the PPG sensor 330 may be configured such that the photodetector 334 of the PPG sensor 330 does not measure light of the first wavelength generated by the LDI sensor 340. This may facilitate simultaneous operation of the PPG sensor and the LDI sensor 340, such that a PPG measurement may be performed concurrently with a LDF measurement while reducing potential crosstalk between the two sensors. For example, the PPG sensor 330 may include a filter (not shown) positioned over the photodetector 334, where the filter is configured to pass light of the second wavelength and filter light of the first wavelength.

In other variations, the PPG sensor 330 may be configured to use the coherent light source 342 of the LDI sensor 340 as its light source. In these variations, the coherent light source 342 may be configured to emit light from the first side 301 of the photonic PCB 300 (e.g., via the first optical aperture 302a), and a portion of this light is returned to and measured by the photodetector 334 of the PPG sensor 330. Accordingly, the coherent light source 342 may be used to perform both PPG and LDF measurements, such that i) a first portion of the emitted signal light emitted from the first optical aperture 302a is collected by the second optical aperture 302b as return signal light as part of the LDF measurement and ii) a second portion of the emitted signal light emitted from the first optical aperture 302a is measured by the photodetector 334 as part of the PPG measurement.

The electrical routing layer(s) of the photonic PCB 300 may be used to power and control the various components of the sensor array (e.g., the LDI sensor 340 and the PPG sensor 330). For example, the electrical routing layer 312 shown in FIG. 3B includes a set of electrical traces 316a-316c. Specifically, the set of electrical traces includes a first electrical trace 316a that is electrically connected to the coherent light source 342, a second electrical trace 316b that is electrically connected to the photodetector 334 of the PPG sensor 330, and a third electrical trace 316c that is electrically connected to the light source 336 of the PPG sensor 330. While not shown in FIG. 3B, the set of electrical traces may include a set of traces that is electrically connected to the pair of photodetectors 348a-348b of the LDI sensor 340. These electrical traces may electrically connect the sensors of the sensor array to a controller (e.g., controller 222), which may facilitate operation of these sensors to perform PPG and/or LDF measurements as described herein.

Similarly, the photonic PCB 300 may further include one or more conductive vias to provide electrical connections through other layers of the photonic PCB 300. For example, FIG. 3B depicts a set of conductive vias 318a-318c that includes i) a first conductive via 318a that extends through the second insulating layer 314b to electrically connect the first electrical trace 316a to the coherent light source 342, ii) a second conductive via 318b that extends through the first insulating layer 314a to electrically connect the second electrical trace 316b to the photodetector 334 of the PPG sensor 330, and iii) a third conductive via 318c that extends through the first insulating layer 314a to electrically connect the third electrical trace 316c to the light source 336 of the PPG sensor 330. It should be appreciated that the electrical traces 316a-316c and conductive vias 318a-318c depicted in FIG. 3B are illustrative examples, and that a wide range of electrical traces and conductive vias may be formed within the photonic PCB 300 to facilitate electrical connection with the various components of the photonic PCB 300.

When a photonic PCB as described herein includes a set of outcouplers to couple light into and/or out of an optical routing layer, each outcoupler may be configured in any suitable manner. For example, in the variation of the photonic PCB 300 shown in FIGS. 3A-3B, each outcoupler of the set of outcouplers 360a-360c is defined by a corresponding angled waveguide facet. In these instances, a portion of a waveguide may be formed with an angled facet that defines the outcoupler, such that light incident on the facet reflects and changes direction. For example, a corresponding angled facet (also indicated by label 360a) of the second waveguide 351 may define the first outcoupler 360a. Accordingly, light traveling in the second waveguide 351 may, when incident on the angled facet of the second waveguide 351, be redirected out of the plane of the optical routing layer 310, such that it exits the photonic PCB through the first optical aperture 302a as emitted signal light. Similarly, a corresponding angled facet (also indicated by label 360b) of the fourth waveguide 353 may define the second outcoupler 360b. Light entering the second optical aperture 302b may, when incident on the angled facet of the fourth waveguide 353, be redirected into the plane of the optical routing layer 310 to enter the fourth waveguide 353. Additionally, a corresponding angled facet of the fifth waveguide 354 may define the third outcoupler 360c, and may redirect the first and second interference signals toward the pair of photodetectors 348a-348b. In some variations, the angled facet may be coated with a reflective material, such as a metal, which may facilitate the reflection of light that is incident on the angled facet.

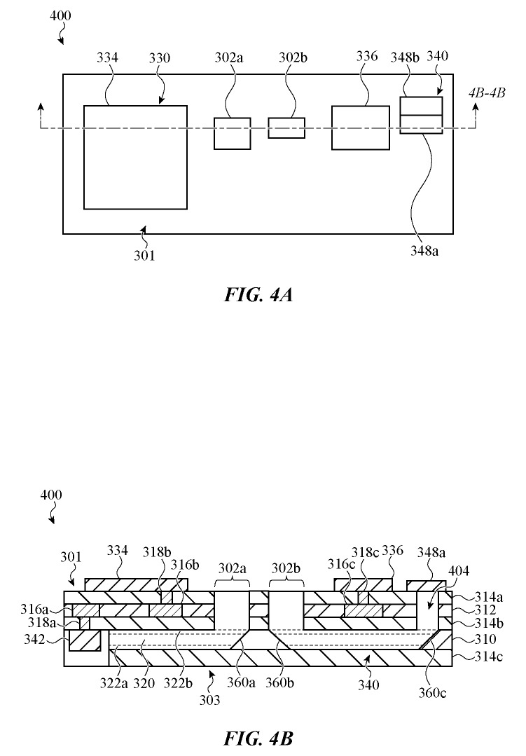

In some variations, one or more outcouplers of a photonic PCB may be defined by corresponding prisms (also referred to herein as a prism outcouplers) that are positioned in the optical routing layer. For example, FIG. 5 shows a cross-sectional side view of a portion of a photonic PCB 500 that includes a set of prism outcouplers. Specifically, the photonic PCB 500 includes an optical routing layer 510 and a first set of layers 511 positioned above the optical routing layer 510, such as described herein with respect to the photonic PCB 300 of FIGS. 3A-3C. In some variations, the photonic PCB 500 may further include a second set of layers 513 positioned below the optical routing layer 510. The photonic PCB 500 includes a set of prism outcouplers, each of which includes a prism having an angled facet that defines a corresponding outcoupler of the photonic PCB 500. In some variations, the angled facet may be formed from or coated with a reflective material, such as a metal, to facilitate redirecting light incident on the angled facet.

For example, a first prism outcoupler may include a first prism 506 that define a first outcoupler. The first prism 506 may redirect signal light 560 carried by a first waveguide 551 defined in the optical routing layer 510. Specifically, the first prism 506 may include a corresponding angled facet 507 that is positioned to reflect and redirect light incident thereon. Signal light 560 traveling in the first waveguide 551 (e.g., in the plane of the optical routing layer 510) is redirected to pass through a first optical aperture 502a that extends through the first set of layers 511. In this way, the first prism outcoupler couples the signal light 560 out of the optical routing layer 510 toward a first side of the photonic PCB 500. In some variations, the first outcoupler 360a of the photonic PCB 300 of FIGS. 3A-3C may alternatively include a prism outcoupler configured in this manner to emit the emitted signal light from the first side 301 of the photonic PCB 300.

Similarly, a second prism outcoupler may include a second prism 508 that defines a second outcoupler. The second prism 508 may redirect return signal light 562 that is received through a second optical aperture 502b that extends through the first set of layers 511. The second prism 508 may include an angled facet 509 that is positioned to redirect light incident thereon. Return signal light 562 that enters the second optical aperture 502b may be directed by the angled facet 509 toward a second waveguide 553 to couple the return signal light 562 into the optical routing layer 510 via the second waveguide 553. In some variations, the second outcoupler 360b of the photonic PCB 300 of FIGS. 3A-3C may alternatively include a corresponding prism outcoupler configured in this manner to collect the return signal light from the first side 301 of the photonic PCB 300. Additionally or alternatively, the third outcoupler 360c of the photonic PCB 300 of FIGS. 3A-3C may alternatively include a corresponding prism outcoupler configured to redirect the first and second interference signals toward the set of photodetectors 348a-348b.

In some variations, a prism may be configured to define both an interferometer of an LDI sensor and one or more outcouplers of a photonic PCB. These prisms, also referred to herein as prism interferometers, may be configured to receive reference light and return signal light of an LDI sensor and to generate a set of interference signals therefrom. For example, FIGS. 6A and 6B show a variation of a photonic PCB 600 that includes an LDI sensor 605 having a prism interferometer 620. Specifically, the photonic PCB 600 includes an optical routing layer 610, a first set of layers 611 positioned above the optical routing layer 610, and a second set of layers 613 positioned below the optical routing layer 610, such as described herein with respect to the photonic PCB 300 of FIGS. 3A-3C. The optical routing layer 610 may define at least a first waveguide 650 that is optically connected each of a second waveguide 651 and a third waveguide 652. The second and third waveguides 651, 652 may concurrently receive signal light 660 and reference light 664, respectively, that are generated during a LDF measurement.

The photonic PCB 600 may be configured to emit the signal light 660 from a first side of the photonic PCB 600 and collect return signal light 662 at the first side of the photonic PCB 600. Specifically, the photonic PCB 600 may include a first set of optical apertures 602a-602b (including a first optical aperture 602a and a second optical aperture 602b), each of which extends through the first set of layers 611, such as described in more detail herein. The photonic PCB 600 may include a first outcoupler (e.g., defined by a prism outcoupler 606 such as shown in FIG. 6B, by an angled waveguide facet outcoupler 360a such as shown in FIG. 3B, or the like) that directs the signal light 660 out of the optical routing layer 610 via the first optical aperture 602a as emitted signal light.

The photonic PCB 600 is further configured to collect return signal light 662 through the second optical aperture 602b, such that the return signal light 662 is incident on the prism interferometer 620. The prism interferometer 620 is configured to act as a beamsplitter to receive and split each of the return signal light 662 and the reference light 664. Specifically, the prism interferometer 620 includes a set of facets that includes a first facet 621 that is optically coupled to the second optical aperture 602b, such that return signal light 662 that enters the photonic PCB 600 via the second optical aperture 602b enters the prism interferometer 620 via the first facet 621. In this way, the first facet 621 of the prism interferometer 620 acts as a first input to receive return signal light 662 and the prism interferometer 620 acts as an outcoupler to couple the return signal light 662 into the optical routing layer 610. The set of facets further includes a second facet 622 that is optically connected to the third waveguide 652, such that reference light 664 traveling through the third waveguide 652 may enter the prism interferometer 620 via the second facet 622. Accordingly, the second facet 622 may act as a second input of the prism interferometer 620 to receive reference light 664.

The set of facets further includes a third facet 623 that is positioned internally within the prism interferometer 620. For example, the prism interferometer 620 may be formed from multiple individual prisms, such that the third facet 623 is formed from an interface between two individual prisms of the prism interferometer 620. In some of these variations, the third facet 623 may be formed from a coating (e.g., an adhesive) positioned between two individual prisms of the prism interferometer 620. The third facet 623 is positioned to receive and split light that enters prism interferometer 620 through the first and second facets 621, 622. Specifically, the prism interferometer 620 is configured such that return signal light 662 entering the first facet 621 is incident on the third facet 623. Return signal light 662 incident on the third facet 623 is split into a first portion that passes through the third facet 623 and a second portion that reflects off of the third facet 623.

Similarly, the prism interferometer 620 is configured such that reference light 664 entering the second facet 622 is incident on the third facet 623, and the third facet 623 splits the reference light 664 into a first portion that reflects off of the third facet 623 and a second portion that passes through the third facet 623. When the prism interferometer 620 simultaneously receives the return signal light 662 and the reference light 664, the first portion of the return signal light 662 interferes with the first portion of the reference light 664 to generate a first interference signal 666. Similarly, the second portion of the return signal light 662 interferes with the second portion of the reference light 664 to generate a second interference signal 668.

The prism interferometer 620 is further configured to emit the first and second interference signals 666, 668, and thus may act as an outcoupler to couple the interference signals from the optical routing layer 610. For example, the prism interferometer may further include a fourth facet 624 that faces opposite the first facet 621, and the prism interferometer 620 may emit the first interference signal 666 from the fourth facet 624. Specifically, the third facet 623 may pass the first portion of the return signal light 662 toward the fourth facet 624, and may redirect the first portion of the reference light 664 toward the fourth facet 624.

In some variations, the prism interferometer 620 is further configured to emit the second interference signal 668 from the fourth facet 624. In these variations, the prism interferometer 620 may further include a fifth facet 625 that is angled to redirect the second interference signal 668 toward the fourth facet 624. Specifically, the third facet may redirect the second portion of the return signal light 662 toward the fifth facet 625 and the may pass the second portion of the reference light 664 toward the fifth facet 625, such that the second interference signal 668 is incident on and reflected by the fifth facet 625 toward the fourth facet 624. In these variations, each of the first and second interference signals 668 may be emitted toward a second side of the photonic PCB 600.

For example, the photonic PCB 600 may include a second set of optical apertures 603a-603b that extend through the second set of layers 613. The second set of optical apertures 603a-603b may include a third optical aperture 603a and a fourth optical aperture 603b, each of which is positioned over the fourth facet 624 of the prism interferometer 620. The LDI sensor 605 may include a pair of photodetectors 648a-648b positioned on the second side of the photonic PCB 600, such that a first photodetector 648a is positioned over the third optical aperture 603a and a second photodetector 648b is positioned over the fourth optical aperture 603b. Accordingly, the first interference signal 666 may be emitted through the third optical aperture 603a and may be measured by the first photodetector 648a. The second interference signal 668 may be emitted through the fourth optical aperture 603b and may be measured by the second photodetector 648b.

It should be appreciated that in some instances, the sensor array of a photonic PCB may include multiple LDI sensors and/or multiple PPG sensors. In some variations in which a photonic PCB includes multiple LDI sensors, some or all of the LDI sensors may share a common light source. For example, FIGS. 7A and 7B show variations of sensor arrays in which multiple LDI sensors share a common light source. For example, FIG. 7A shows an example of a sensor array 700 that includes a plurality of LDI sensors that share a common light source 703. The sensor array 700 may be incorporated into a photonic PCB having an optical routing layer, such as described in more detail herein. Specifically, the sensor array 700 includes the common light source 703 and a common waveguide 701. The common waveguide 701 may be defined in an optical routing layer of a photonic PCB (e.g., photonic PCB 300) and may be positioned to receive light generated by the common light source 703. The sensor array 700 defines a plurality of LDI sensors 705a-705c, each of which may be operated to perform a corresponding LDF measurement using light generated by the common light source 703.

The common waveguide 701 may split light received by common light source 703, such that each LDI sensor of the plurality of LDI sensors 705a-705c receives a corresponding portion of the light generated by the common light source 703. In some variations, such as shown in FIG. 7A, the common waveguide 701 may be tapered such that its width increases in a direction away from the common light source 703. This may facilitate splitting received from the common light source 703 between the plurality of LDI sensors 705a-705c.

Specifically, each LDI sensor of the plurality of LDI sensors 705a-705c includes the common light source 703, the common waveguide 701, a corresponding set of waveguides optically connected to the common waveguide 701, a corresponding interferometer, and a corresponding pair of photodetectors. Each LDI sensor is configured to generate its own signal light and reference light using light received by the common waveguide, and may use this light to generate a corresponding pair of interference signals. Accordingly, the corresponding pair of photodetectors for a given LDI sensor of the plurality of LDI sensors 705a-705c may measure the corresponding pair of interference signals as part of a LDF measurement.

Each LDI sensor may be configured in any manner as described herein with respect to FIGS. 3A-6B. In the variation of the sensor array 700 shown in FIG. 7A, the corresponding interferometer each of the LDI sensors 705a-705c includes a waveguide interferometer, such as described herein with respect to the LDI sensor 340 of FIGS. 3A-3C. For example, a first LDI sensor 705a includes a corresponding first set of waveguides 702a-702b optically connected to the common waveguide 701. The first set of waveguides 702a-702b includes a first waveguide 702a that acts a first signal path for the first LDI sensor 705a to receive signal light from the common waveguide. The first set of waveguides 702a-702b further includes a second waveguide 702b that acts as a reference path for the first LDI sensor 705a to receive reference light from the common waveguide.

The first LDI sensor 705a includes a corresponding first set of outcouplers 704a-704c to redirect light within the first LDI sensor 705a. The first set of outcouplers 704a-704c may include a first outcoupler 704a that is optically connected to the first waveguide 702a of the first set of waveguides 702a-702b. The first outcoupler 704a may emit the emitted signal light from the first LDI sensor 705a (e.g., via a corresponding optical aperture of the photonic PCB). The first set of outcouplers 704a-704c may also include a second outcoupler 704b configured to collect return signal light for the first LDI sensor 705a (e.g., via a corresponding optical aperture of the photonic PCB). The first LDI sensor 705a includes a corresponding first interferometer 706, which in the variation shown in FIG. 7A is configured as a waveguide interferometer that is optically coupled to each of the second waveguide 702b and the second outcoupler 704b of the first LDI sensor 705a.

Accordingly, the interferometer 706 of the first LDI sensor 705a may receive the corresponding reference light and return signal light and generate a corresponding first pair of interference signals. The first LDI sensor 705a includes a corresponding first pair of photodetectors 708a-708b that is configured to measure the first pair of interference signals. Specifically, a first photodetector 708a of the first pair of photodetectors 708a-708b measures a first interference signal of the first pair of interference signals and a second photodetector 708b of the first pair of photodetectors 708a-708b measures a second interference signal of the second pair of interference signals. For example, the first set of outcouplers 704a-704c may include a third outcoupler 704c that is optically connected to the interferometer 706 and configured to redirect the first pair of interference signals toward the first pair of photodetectors 708a-708b (e.g., via a corresponding optical aperture of the photonic PCB).

The plurality of LDI sensors 705a-705c may include a second LDI sensor 705b that may be configured in the same manner as the first LDI sensor 705a. The second LDI sensor 705b may include a corresponding second set of waveguides 712a-712b optically connected to common waveguide 701 and configured to receive signal light and reference light for the second LDI sensor 705b. The second LDI sensor 705b may include a corresponding second interferometer 716 that is configured to generate a corresponding second pair of interference signals, and may further include a second pair of photodetectors 718a-718b that is positioned to measure the second pair of interference signals. Additionally, the second LDI sensor 705b may include a corresponding second set of outcouplers 714a-714c that may operate as described herein with respect to the first set of outcouplers 704a-704c to route light relative to the second LDI sensor 705b.

In some variations, the plurality of LDI sensors 705a-705c may include one or more additional LDI sensors, such as a third LDI sensor 705c. The third LDI sensor 705c may include a corresponding third set of waveguides 722a-722b optically connected to common waveguide 701 and configured to receive signal light and reference light for the third LDI sensor 705c. The third LDI sensor 705c may include a corresponding third interferometer 726 that is configured to generate a corresponding third pair of interference signals, and may further include a third pair of photodetectors 728a-728b that is positioned to measure the third pair of interference signals. Additionally ,the third LDI sensor 705c may include a corresponding third set of outcouplers 724a-724c that may operate as described herein with respect to the first set of outcouplers 704a-704c to route light relative to the third LDI sensor 705c.

Overall, each LDI sensor of the plurality of LDI sensors 705a-705c may be configured to emit and collect signal light from different locations of the sensor array 700, which may allow for the sensor array 700 to perform LDF measurements at different skin regions of a user. Each of the LDI sensors may be associated with a corresponding separation distance that represents a distance between where the signal light is emitted from the sensor array 700 and where the return signal light is collected by the sensor array 700 for that LDI sensor. For example, some or all of the LDI sensors 705a-705b may have a common separation distance. In these variations, the first and second outcouplers of each LDI sensor may be separated by a common distance (e.g., a first distance between the first outcoupler 704a and the second outcoupler 704b of the first LDI sensor 705a may be the same as a second distance between the first outcoupler 714a and the second outcoupler 714b of the second LDI sensor 705b). In some variations, some or all of the LDI sensors 705a-705b may have different separation distances (e.g., the first distance between the first outcoupler 704a and the second outcoupler 704b of the first LDI sensor 705a may different than the second distance between the first outcoupler 714a and the second outcoupler 714b of the second LDI sensor 705b).

FIG. 7B shows another variation of a sensor array 730 that includes multiple sensor subarrays, where each subarray shares a corresponding common light source. Specifically, the sensor array 730 includes i) a first sensor subarray 740 that includes a first plurality of LDI sensors that share a first common light source 743 and a first common waveguide 741, and ii) a second sensor subarray 760 that includes a second plurality of LDI sensors that share a second common light source 763 and a second common waveguide 761. During operation of the sensor array 730, the first and second sensor subarrays 740, 760 may be operated simultaneously or sequentially as may be desired.

Each of the LDI sensors of the sensor array 730 may be configured in any manner as described herein with respect to the sensor array 700 of FIG. 7A. In the variation of the sensor array 730 shown in FIG. 7B, each of the LDI sensors are configured to include a corresponding prism interferometer. For example, the first sensor subarray 740 includes a first LDI sensor that includes a corresponding first set of waveguides 742a-742b, a first outcoupler 744, a first prism interferometer 746, and a first set of photodetectors (not shown). A first waveguide 742a of the first set of waveguides 742a-742b may act as a signal path to receive signal light from the first common waveguide 741 and may be optically connected to the first outcoupler 744 to emit the signal light from the first LDI sensor. The second waveguide 752b of the first set of waveguides 742a-742b may act as a reference path to receive reference light from the first common waveguide 741, and may be optically connected to the first prism interferometer 746. Accordingly, the first prism interferometer 746 may receive reference light and return signal light, and may generate a first pair of interference signals.

Similarly, the first sensor subarray 740 may include a second LDI sensor that includes a second set of waveguides 752a-752b, a second outcoupler 754, a second prism interferometer 756, and a second pair of photodetectors (not shown). The first and second sets of waveguides of the first sensor subarray 740 may be connected to the first common waveguide 741 in any suitable arrangement. For example, in the variation shown in FIG. 7B, the second waveguides 742b, 752b of the first and second sets of waveguides may each be positioned between the first waveguides 742a, 752a of the first and second sets of waveguides. The LDI sensors of the second sensor subarray 760 may be configured in a similar manner. For example, the second sensor subarray 760 includes i) a third LDI sensor that includes a second set of waveguides 762a-762b, a second outcoupler 764, a second prism interferometer 766, and a second pair of photodetectors (not shown), and ii) a fourth LDI sensor that includes a second set of waveguides 772a-772b, a second outcoupler 774, a second prism interferometer 776, and a second pair of photodetectors (not shown).

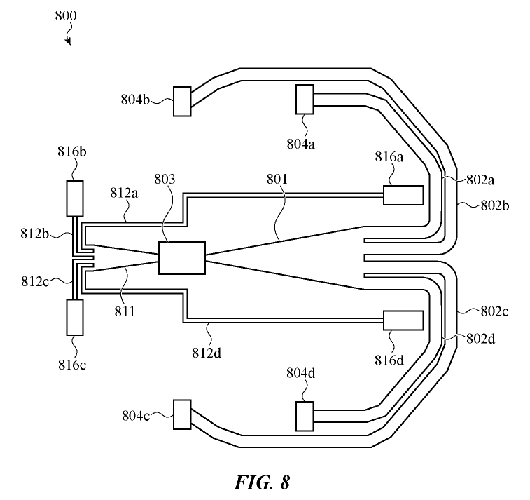

In the variations of the sensor arrays 700, 730 of FIGS. 7A-7C, each LDI sensor receives signal light and reference light from the same common waveguide. In other variations, a sensor array may include a plurality of LDI sensors that each receive signal light and reference light from different common waveguides. For example, FIG. 8 shows a variation of sensor array 800 that includes a plurality of LDI sensors that share a common light source 803, a first common waveguide 801, and a second common waveguide 811. In these variations, the common light source 803 is configured to generate two different light beams, such that the first common waveguide 801 receives a first light beam from the common light source 803 and the second common waveguide 811 receives a second light beam from the common light source 803. Light received by the first common waveguide 801 may be used to generate corresponding signal light for each of the plurality of LDI sensors, and light received by the second common waveguide 811 may be used to generate reference light for each of the plurality of LDI sensors.

For example, a first plurality of waveguides 802a-802d may be optically connected to the first common waveguide 801 and a second plurality of waveguides 812a-812d may be optically connected to the second common waveguide 811. Each LDI sensor may include a corresponding waveguide of the first plurality of waveguides 802a-802d and a corresponding waveguide of the second plurality of waveguides 812a-812d. For example, a first LDI sensor includes a first waveguide 802a of the first plurality of waveguides 802a-802d and a first waveguide 812a of the second plurality of waveguides 812a-812d, a second LDI sensor includes a second waveguide 802b of the first plurality of waveguides 802a-802d and a second waveguide 812b of the second plurality of waveguides 812a-812d, and so on. The sensor array 800 may include a plurality of outcouplers 804a-804d configured to emit signal light for each LDI sensor (e.g., a first outcoupler 804a emits signal light from the first waveguide 802a of the first plurality of waveguides 802a-802d for the first LDI sensor, a second outcoupler 804b emits signal light from the second waveguide 802b of the first plurality of waveguides 802a-802d for the first LDI sensor, and so on). Similarly, the sensor array 800 may include a plurality of interferometers (e.g., a plurality of prism interferometers 816a-816d) configured to generate a corresponding pair of interference signals for each LDI sensor. For example, the first LDI sensor may include a first prism interferometer 816a, the second LDI sensor may include a second prism interferometer 816b, and so on.

In instances where an LDI sensor receives signal light and reference light from different outputs of a light source, the reference light may be modulated independently of the signal light. For example, the sensor array 800 may include a phase modulator configured to modulate the phase of second light beam generated by the common light source 803, which may modulate the phase of the reference light for each of the plurality of LDI sensors. During a LDF measurement, a phase of the reference light may be modulated according to a predetermined frequency, which may also modulate the interference signals generated by the LDI sensor. This modulation may be taken into account when analyzing the LDF measurement signals, and may make the LDF measurements more robust to motion of the LDI sensor relative to a user.

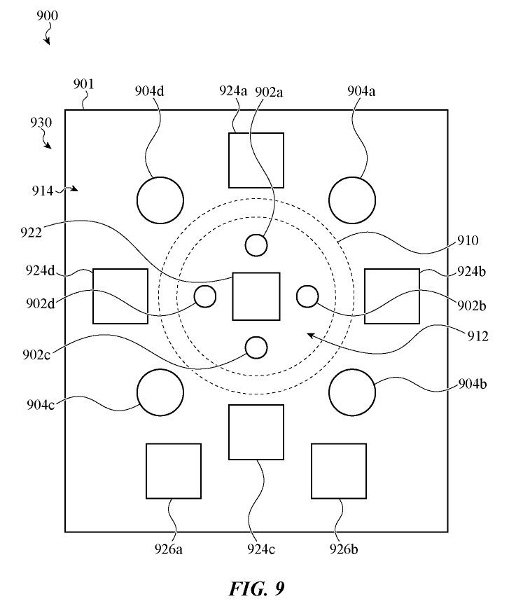

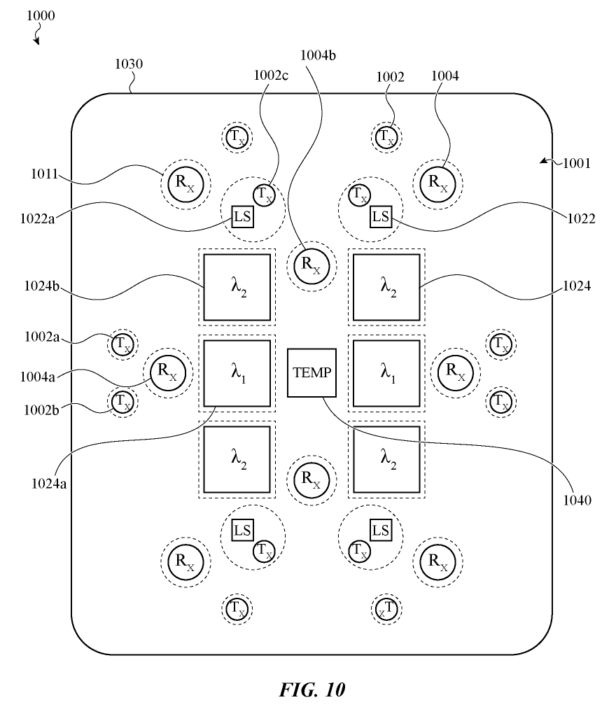

The sensors of the sensor arrays described herein may be arranged in any suitable manner. FIGS. 9 and 10 depict different example arrangements of sensors of the optical sensor modules described herein. For example, FIG. 9 shows a top view of a portion of an optical sensor module 900 that includes a plurality of LDI sensors and a plurality of PPG sensors. Specifically, the optical sensor module 900 includes a photonic PCB 930 having a first side 901. The optical sensor module 900 may include an optical barrier 910 that defines an inner cavity 912 surrounded by and optically isolated from an outer cavity 914. The optical sensor module 900 may be configured to emit light through the inner cavity 912 and to collect light through the outer cavity 914.

For example, the photonic PCB 930 may include a first set of optical apertures 902a-902d that are positioned within the inner cavity 912 and configured to emit light from the first side 901 of the photonic PCB 930. Each optical aperture 902a-902d may be positioned over a corresponding outcoupler (not shown), which may be used to redirect light that is generated by a corresponding light source and carried by an optical routing layer of the photonic PCB 930. In the variation shown in FIG. 9, the first set of optical apertures 902a-902d includes a plurality of optical apertures. While the first set of optical apertures 902a-902d is shown in FIG. 9 as including four optical apertures (e.g., a first optical aperture 902a, a second optical aperture 902b, a third optical aperture 902c, and a fourth optical aperture 902d), it should be appreciated that the first set of optical apertures 902a-902d of may include more or fewer optical apertures as may be desired.

In instances where the first set of optical apertures 902a-902d includes a plurality of optical apertures, some or all of the optical apertures 902a-902d may receive light generated by a common light source. For example, each of the optical apertures 902a-902d may receive light generated by a common light source. In these variations, all of the optical apertures 902a-902d may emit light simultaneously. In other variations, two more of the optical apertures 902a-902d be configured to receive light from different light sources. In these instances, the optical sensor module 900 may be operated such that different optical apertures (or groups thereof) of the first set of optical apertures 902a-902d emit light at different times.

The photonic PCB 930 may further include a second set of optical apertures 904a-904d positioned within the outer cavity 914 and configured to collect light that is incident on the first side 901 of the photonic PCB 930. Each optical aperture 904a-904d may be positioned over a corresponding outcoupler (not shown), which may include a prism interferometer as described herein and which may be couple light into an optical routing layer of the photonic PCB 930. In the variation shown in FIG. 9, the second set of optical apertures 904a-904d includes a plurality of optical apertures. While the second set of optical apertures 904a-904d is shown in FIG. 9 as including four optical apertures (e.g., a first optical aperture 904a, a second optical aperture 904b, a third optical aperture 904c, and a fourth optical aperture 904d), it should be appreciated that the second set of optical apertures 904a-904d of may include more or fewer optical apertures as may be desired.

Each optical aperture of the second set of optical apertures 904a-904d may be associated with a different LDI sensor of the optical sensor module 900. Specifically, each LDI sensor may include a corresponding light source, at least one corresponding optical aperture of the first set of optical apertures 902a-902d (e.g., to emit emitted signal light from the photonic PCB 930), a corresponding optical aperture of the second set of optical apertures 904a-904d (e.g., to collect return signal light), a corresponding interferometer, and a corresponding pair of detectors (not shown). For each LDI sensor, the photonic PCB may include additional optical components (e.g., waveguides, outcouplers, or the like) as needed to route signal light and reference light within the LDI sensor and to route a corresponding pair of interference signals to the corresponding pair of photodetectors. The pairs of photodetectors corresponding to the LDI sensors are not shown in FIG. 9, and may each be positioned on a second side (not shown) of the photonic PCB 930 (such as described herein with respect to the photonic PCB 300 of FIGS. 3A-3C). In other variations, however, some or all of the photodetectors of the LDI sensors may be positioned on the first side 901 of the photonic PCB 930 (such as described herein with respect to the photonic PCB 400 of FIGS. 4A and 4B).

Accordingly, to operate each LDI sensor, the photonic PCB may emit signal light through one or more of the first set of optical apertures 902a-902d and collect return signal light through a corresponding optical aperture of the second set of optical apertures 904a-904d. The corresponding interferometer may interfere the return signal light with reference signal light (such as described herein with respect to FIGS. 3A-8) to generate the corresponding pair of interference signals, which may be measured by the corresponding pair of photodetectors. The photonic PCB 930 is shown in FIG. 9 as including four LDI sensors (e.g., a first LDI sensor that collects return signal light through the first optical aperture 904a in the outer cavity 914, a second LDI sensor that collects return signal light through the second optical aperture 904b in the outer cavity 914, a third LDI sensor that collects return signal light through the third optical aperture 904c in the outer cavity 914, and a fourth LDI sensor that collects return signal light through the fourth optical aperture 904d in the outer cavity 914).

The optical sensor module 900 may include one or more sets of PPG sensors that are incorporated into the photonic PCB 930. For example, in the variation shown in FIG. 9, the optical sensor module 900 includes a set of light sources that are mounted on the first side 901 of the photonic PCB 930 and positioned in the inner cavity 912. In some variations, each light source of the set of light sources is a light emitting diode. In the variation shown in FIG. 9, the first set of light sources includes a single light source 922, though in other variations the first set of light sources may include a plurality of light sources positioned within the inner cavity 912. Each light source of the first set of light sources may act as a light source for one or more PPG sensors of the optical sensor module 900.

Additionally, the optical sensor module 900 may include a first set of photodetectors 924a-924d that are mounted to the first side 901 of the photonic PCB 930 within the outer cavity 914. In these variations, the optical sensor module 900 may include a first set of PPG sensors, each of which includes a corresponding photodetector of the first set of photodetectors 924a-924d. For example, in the variation shown in FIG. 9, the first set of photodetectors 924a-924d includes a plurality of photodetectors (e.g., a first photodetector 924a, a second photodetector 924b, a third photodetector 924c, and a fourth photodetector 924d) and a corresponding plurality of PPG sensors.

The first set of photodetectors (and thus the first set of PPG sensors) may be configured to measure light emitted by one or more light sources of the first set of light sources. For example, in the variation shown in FIG. 9, each of the first set of PPG sensors may share the light source 922, such that each of the PPG sensors is configured to measure light emitted form the light source 922. In other variations, each PPG sensor of the first set of PPG sensors is configured to measure light generated by a different corresponding light source of the first set of light sources.

In some variations, the first set of PPG sensors may be configured such that their corresponding photodetectors (e.g., the first set of photodetectors 924a-924d) do not measure light emitted by the first set of optical apertures 902a-902d. For example, the optical sensor module 900 may be configured to emit light of a first wavelength (e.g., an infrared wavelength) from the first set of optical apertures 902a-902d, and each light source of the first set of light sources is configured to emit a corresponding wavelength of light that is different than the first wavelength (e.g., the light source 922 may emit a second wavelength, such as a green wavelength, that is different than the first wavelength). The optical sensor module 900 may include a corresponding filter positioned over each of the first set of photodetectors 924a-924d, where the filter is configured to filter out light of the first wavelength.

In some instances, the first set of photodetectors 924a-924b and the second set of optical apertures 904a-904d may be radially arranged around the optical barrier 910 and the inner cavity 912, such that the photodetectors of the first set of photodetectors 924a-924d alternate with the optical apertures of the second set of optical apertures 904a-904d. For example, the first optical apertures 904a of the second set of optical apertures 904a-904d is positioned between the first and second photodetectors 924a, 924b of the first set of photodetectors 924a-924d, the second photodetector 924b is positioned between the first and second optical apertures 904a, 904b, and so on. Collectively, the first set of PPG samples may collect light from similar skin regions as the plurality of LDI sensors.

In some variations, the optical sensor module 900 may include a second set of PPG sensors that utilize light emitted from the first set of optical apertures 902a-902d. For example, the optical sensor module 900 may include a second set of photodetectors 926a-926b, each of which is positioned on the first side 901 of the photonic PCB 930. In these variations, the optical sensor module 900 may include a second set of PPG sensors, each of which includes a corresponding photodetector of the second set of photodetectors 926a-926d. For example, in the variation shown in FIG. 9, the second set of PPG sensors includes a first PPG sensor corresponding to a first photodetector 926a of the second set of photodetectors 926a-926b, and further includes a second PPG sensor corresponding to a second photodetector 926b of the second set of photodetectors 926a-926b. Each PPG sensor of the second set of PPG sensors is configured to measure light emitted from one or more of the first set of optical apertures 902a-902d. For example, in variations in which the optical sensor module 900 is configured to emit a first wavelength of light from the first set of optical apertures 902a-902d, each of the second set of photodetectors 926a-926b may be configured to measure light of the first wavelength.

FIG. 10 shows another variation of an optical sensor module 1000 in which different components may be positioned in different cavities. The optical sensor module 1000 includes a photonic PCB 1030 having a first side 1001. The optical sensor module 1000 may further include an optical barrier positioned over the first side 1001 of the photonic PCB 1030, and that defines a plurality of cavities 1011 that are optically isolated by each other. While a single cavity 1011, it should be appreciated that additional shapes defined by a dashed line in FIG. 10 represent other cavities of the optical sensor module 1000. The first side 1001 of the photonic PCB 1030 may include a first plurality of optical apertures 1002, each of which is represented by a circle and labeled Tₓ in FIG. 10. Each optical aperture of the first plurality of optical apertures 1002 (also referred to herein as “launch optical apertures”) is configured to emit light from the first side 1001 of the photonic PCB 1030. Similarly, the first side 1001 of the photonic PCB 1030 may include a second plurality of optical apertures 1004, each of which is represented by a circle and labeled Rₓ in FIG. 10. Each optical aperture of the first plurality of optical apertures 1002 (also referred to herein as “collection optical apertures”) is configured to collect light that is incident on the first side 1001 of the photonic PCB 1030. Accordingly, the optical sensor module 1000 may include a plurality of LDI sensors (such as described in more detail herein) that includes at least one corresponding launch optical aperture 1002 and a corresponding collection optical aperture 1004.

Additionally, the optical sensor module 1000 may include a plurality of light sources 1022, each of which is represented by a square and labeled LS in FIG. 10. Each light source 1022 may be positioned on the first side 1001 of the photonic PCB 1030 and configured to emit light away from the first side 1001 of the photonic PCB 1030. Each light source 1022 may emit light that may be detected by a photodetector as part of a corresponding PPG sensor. For example, the optical sensor module 1000 may include a plurality of photodetectors 1024, each of which is represented by a square and labeled λ_{N}, where N indicates a wavelength sensitivity of the photodetector 1024. For example, the optical sensor module 1000 may be configured to emit a first wavelength of light λ₁ from the launch optical apertures 1002, and may be configured to emit a second wavelength of light λ₂ from the light sources 1022. Accordingly, a first photodetector 1024a (labeled λ₁) may be configured to measure the first wavelength, but not the second wavelength (e.g., a filter may be positioned over the first photodetector 1024a that filters out light of the second wavelength). Similarly, a second photodetector 1024b (labeled λ₂) may be configured to measure the second wavelength, but not the first wavelength (e.g., a filter may be positioned over the second photodetector 1024b that filters out light of the second wavelength).

In some instances, the optical sensor module 1000 may include an LDI sensor and a PPG sensor that utilize the same illumination. In one example, an LDI sensor may include a corresponding set of launch optical apertures (e.g., including launch optical apertures 1002a, 1002b in FIG. 10) and a collection optical aperture 1004a, and a PPG sensor may include the same set of launch optical apertures and photodetector 1024a. Accordingly, light emitted from the set of launch optical apertures 1002a, 1002b may, after returning to the optical measurement system may be i) collected by the collection optical aperture 1004a as part of a LDF measurement performed by the LDI sensor and ii) measured by the photodetector 1024a as part of a PPG measurement performed by the PPG sensor. In the variation shown in FIG. 10, the photodetector 1024a is positioned closer to a center of the optical sensor module 1000 than the collection optical aperture 1004a. This may prioritize reducing ambient light received by the PPG sensor (e.g., components positioned closer to the center of the optical sensor module 1000 may receive less ambient light when an electronic device incorporating the optical sensor module 1000 is worn by a user). Additionally, in the variation shown in FIG. 10, the collection optical apertures 1004a is positioned between the photodetector 1024a and the set of launch optical apertures 1002a, 1002b, which may promote sampling of similar regions of a user’s skin by the PPG sensor and the LDI sensor. Each of the set of launch optical apertures 1002a, 1002b, the collection optical aperture 1004a, and the photodetector 1024a is shown in FIG. 10 as being positioned in a different corresponding cavity 1011 of the optical sensor module 1000.

In another example, an LDI sensor may include a corresponding set of launch optical apertures (e.g., including launch optical apertures 1002c in FIG. 10) and a collection optical aperture 1004b, and a PPG sensor may include a light source 1022a and a photodetector 1024b. In these examples, the launch optical aperture 1002c and the light source 1022a may be positioned in the same cavity 1011, whereas the collection optical aperture 1004b and the photodetector 1024b are positioned in different cavities 1011 within the optical sensor module 1000. It should be appreciated that a sensor array of the optical sensor module 1000 may include a wide variety of arrangements of LDI sensors and PPG sensors as may be desired.

Additionally, the optical sensor module 1000 may include one or more additional sensors, such as a temperature sensor 1040. For example the temperature sensor 1040 may be positioned on or above the first side 1001 of the photonic PCB 1030, and may be configured to measure a temperature at a corresponding portion of the optical sensor module 1000. In instances where the optical sensor module 1000 (e.g., via a cover of the optical sensor module 1000) is positioned in contact with a user’s skin, the temperature measured by the temperature sensor 1040 may be indicative of the local skin temperature of the user. Temperature measurements performed by the temperature sensor 1040 may be used in analyzing the PPG and/or LDF measurements performed by the optical sensor module 1000. It should be appreciated that the temperature sensor 1040 may be included in any of the optical sensor modules described herein. For example, the optical sensor module 900 of FIG. 9 may include a temperature sensor (not shown), which in some examples may be positioned in the inner cavity 912.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. An optical sensor module comprising:
a photonic printed circuit board having a first side and a second side and comprising a laser doppler interferometry sensor and a photoplethysmogram sensor, wherein:
the laser doppler interferometry sensor comprises:
a light source assembly configured to generate signal light and reference light, wherein the laser doppler interferometry sensor is configured to emit the signal light from the first side of the photonic printed circuit board and collect return signal light through the first side of the photonic printed circuit board;
an interferometer configured to receive the reference light and the return signal light and generate a pair of interference signals; and
a pair of photodetectors positioned to measure the pair of interference signals; and
the photoplethysmogram sensor comprises:
a photodetector positioned on the first side of the first side of the photonic printed circuit board.

2. The optical sensor module of claim 1, wherein the pair of photodetectors are positioned on the first side of the photonic printed circuit board.

3. The optical sensor module of claim 1, wherein the pair of photodetectors are positioned on the second side of the photonic printed circuit board.

4. The optical sensor module of claim 1, wherein the photoplethysmogram sensor comprises a light source positioned on the first side of the photonic printed circuit board.

5. The optical sensor module of claim 1, wherein:
the light source assembly comprises a coherent light source; and
the photoplethysmogram sensor comprises the coherent light source.

6. The optical sensor module of claim 1, wherein the photonic printed circuit board comprises:
an optical routing layer;
a first outcoupler positioned to couple the signal light from the optical routing layer toward the first side; and
a second outcoupler positioned to couple the return signal light into the optical routing layer.

7. The optical sensor module of claim 6, wherein the first outcoupler is a prism outcoupler.

8. The optical sensor module of claim 6, wherein the optical routing layer comprises a prism interferometer that defines the second outcoupler and the interferometer.

9. An optical sensor module comprising:
a photonic printed circuit board defining a common waveguide;
a common light source mounted to the photonic printed circuit board; and
a plurality of laser doppler interferometry sensors, wherein each laser doppler interferometry sensor of the plurality of laser doppler interferometry sensors comprises:
the common waveguide;
the common light source;
a corresponding set of waveguides optically connected to the common waveguide;
a corresponding interferometer; and
a corresponding pair of photodetectors.

10. The optical sensor module of claim 9, wherein the corresponding interferometer for each laser doppler interferometry sensor of the plurality of laser doppler interferometry sensors comprises a prism interferometer.

11. The optical sensor module of claim 9, wherein:
the common waveguide is a first common waveguide;
the photonic printed circuit board defines a second common waveguide; and
each laser doppler interferometry sensor of the plurality of laser doppler interferometry sensors comprises a corresponding waveguide optically connected to the second common waveguide.

12. An optical sensor module comprising:
a cover;
an optical barrier defining an inner cavity and an outer cavity surrounding the inner cavity;
a photonic printed circuit board, wherein the photonic printed circuit board is configured to:
emit light from a first side of the photonic printed circuit board through a first set of optical apertures positioned in the inner cavity; and
receive light received at the first side of the photonic printed circuit board through a second set of optical apertures positioned in the outer cavity;
a set of light sources positioned on the first side of the photonic printed circuit board in the inner cavity; and
a first set of photodetectors positioned on the first side of the photonic printed circuit board in the outer cavity.

13. The optical sensor module of claim 12, wherein:
the first set of optical apertures comprises a plurality of optical apertures.

14. The optical sensor module of claim 12, wherein:
the photonic printed circuit board is configured to emit a first wavelength of light through the first set of optical apertures; and
the set of light sources is configured to emit a second wavelength of light.

15. The optical sensor module of claim 14, wherein:
the first set of photodetectors is configured measure the second wavelength of light.

16. The optical sensor module of claim 15, comprising:
a second set of photodetectors positioned on the first side of the photonic printed circuit board in the outer cavity, wherein the second set of photodetectors is configured measure the first wavelength of light.

17. The optical sensor module of claim 12, wherein the first set of photodetectors and the second set of optical apertures are radially arranged around the optical barrier.

18. The optical sensor module of claim 17, wherein photodetectors of the first set of photodetectors alternate with the optical apertures of the second set of optical apertures.

19. The optical sensor module of claim 12, comprising a set of laser doppler interferometry sensors, wherein each laser doppler interferometry sensor of the set of laser doppler interferometry sensors comprises:
one or more corresponding optical apertures of the first set of optical apertures;
a corresponding optical aperture of the second set of optical apertures;
a corresponding interferometer; and
a corresponding pair of photodetectors.

20. The optical sensor module of claim 19, wherein the corresponding pair of photodetectors for each laser doppler interferometry sensor of the set of laser doppler interferometry sensors is positioned on a second side of the photonic printed circuit board.
